(19) United States
(12) Patent Application Publication
(10) Pub. No.: US 2021/0353705 A1
(43) Pub. Date: Nov. 18, 2021

(54) METHOD AND COMPOSITION FOR RELIEVING FATIGUE AND RESTORING ENERGY

(71) Applicant: India Globalization Capital, Inc., Potomac, MD (US)

(72) Inventors: Ramachandra MUKUNDA, Potomac, MD (US); Jagadeesh Sridhara RAO, Ashburn, VA (US)

(73) Assignee: India Globalization Capital, Inc., Potomac, MD (US)

(21) Appl. No.: 17/282,332

(22) PCT Filed: Oct. 4, 2019
(86) PCT No.: PCT/US2019/054858

§ 371 Date: Apr. 1, 2021

Related U.S. Application Data
(60) Provisional application No. 62/741,235, filed on Oct. 4, 2018.

(51) Int. Cl. A61K 36/77; A61K 9/00; A61K 31/05; A61K 31/352; A61K 36/185; A61K 31/522; A61K 31/198; A61K 31/194; A61K 31/455; A61K 33/06; A61K 47/36; A61K 31/375; A61K 47/26; A61P 21/00

(52) U.S. Cl. CPC A61K 36/77; A61K 9/0095; A61K 31/05; A61K 31/352; A61K 36/185; A61K 31/522; A61P 21/00; A61K 31/194; A61K 31/455; A61K 33/06; A61K 47/36; A61K 31/375; A61K 47/26; A61K 31/198

(57) ABSTRACT

Methods and compositions for relieving general and chronic fatigue, restoring energy, rejuvenating sore muscles, and relieving associated pain by administering a composition including caffeine, and a Cannabis component having one or more compounds. An aqueous composition (e.g., an “energy” drink) comprises the caffeine and a Cannabis component selected from the group of cannabidiol (CBD), tetrahydrocannabinol (THC), hemp oil, Cannabis oil, and CBD together with THC. They are dissolved or diluted in water along with optional GRAS ingredients.

### CROSS-REFERENCE TO RELATED APPLICATION

This application claims priority benefit of Provisional Application No. 62/741,235, filed Oct. 4, 2018; the content of which is hereby incorporated herein by reference in its entirety.

### INTRODUCTION

Methods and compositions for relieving general and chronic fatigue, restoring energy, rejuvenating sore muscles, and relieving associated pain by administering a composition including caffeine, and a Cannabis compound.

Background

Fatigue is one of the most widespread symptoms in modern society. This particular illness generally vanishes after taking rest or treating causative diseases, but uncontrolled chronic fatigue raises a variety of problems in physical and social activities. A recent report from the United States reported that untreated chronic fatigue syndrome lead to reduce the workforce productivity by 54% of afflicted individuals leading to 9.1 billion dollars of total productivity loss in the US alone (Son. Review of the prevalence of chronic fatigue worldwide. J Korean Oriental Med 2012; 33(2):25-33).

Various studies have reported that the individual suffering from chronic fatigue due to over extortion at work or physical fatigue due to sport activities, seasonal variations and other reasons seek energy booster in the form of energy drink, or energy drink or energy enhancing pills. Most of the energy drink or energy drinks are intended to improve energy, stamina, athletic performance, and concentration.

Most of the energy drinks are associated with high levels of sugars to provide immediate relief. The long-term consumption of energy drinks containing sugar is associated with poor health (World Health Organization. WHO Calls on Countries to Reduce Sugars Intake among Adults and Children. Geneva: 2015).

The caffeine concentration in energy drink ranges widely from 47 to 80 mg per 8 oz to as high as 207 mg per 2 oz and comes from a number of ingredient sources (Generali. Energy drinks: Food, dietary supplement, or drug? Hosp Pharm 2013; 48(1):5.10.1310/hpj4801-5). Moderate caffeine intake (up to 400 mg/day) is generally considered safe and even beneficial for health among adults (McLellan et al. A review of caffeine's effects on cognitive, physical and occupational performance. Neurosci Biobehav Rev 2016 December; 71:294-312).

The role of the endocannabinoid system (ECS) has been implicated in the development and progression of chronic fatigue syndrome (also known as myalgic encephalomyelitis and post-exertional malaise). Studies have been done demonstrating that the role of ECS in stress, generalized fatigue and post-viral fatigue conditions. Additionally, there have been several studies done on the ECS and related conditions such as chronic stress, fibromyalgia syndrome (FMS), and multiple sclerosis (MS), which list chronic fatigue as one of their primary symptoms.

Caffeine is known to enhance the anti-inflammatory response during running. (Tauler et al. Med Sci

Sports Exercise 2013 July; 45(7):1269-1276). Similar to caffeine, ultralow-doses of THC have been reported to exhibit anti-inflammatory properties (Rock et al. Psychopharmacology 2018 November; 235(11):3259-3271). CBD protects against inflammation induced pain (Philpot et al. Pain 2017 December; 158(12):2442-2451). However, the synergetic effects of cannabinoids in combination with caffeine has not been studied for fatigue.

### SUMMARY

The invention provides a therapeutic drink composition for treating fatigue, sore muscles, and pain associated therewith, which includes: (i) a first component selected from the group consisting of cannabidiol (CBD), tetrahydrocannabinol (THC), hemp oil, Cannabis oil, and CBD together with THC; (ii) caffeine, preferably from a guarana extract; and (iii) a third component comprising at least three members selected from the group consisting of lysine, arginine malate, arginine alpha ketoglutarate, taurine, niacin, magnesium sulfate, multivitamins, vitamin C, sucralose, citric acid, potassium sorbate, and xantham gum; which are all dissolved in reverse osmosis water, with natural flavoring.

The preferred amounts of the components in the energy drink are 0.025% caffeine, 0.5% CBD, and 0.0025% THC with preferred ranges from about 0.01% to about 0.08% of caffeine, from about 0.5% to about 10% (or less than 1%) of CBD, and from about 0.0025% to about 2% (or from about 0.025% to about 0.5%) of THC in weight/volume percent. Caffeine from about 10 mg to about 80 mg and CBD from about 500 mg to about 10 gm and/or THC from about 2.5 mg to about 2 gm may be present per 100 ml of the drink composition.

In addition to above ingredients, the preferred ranges of other components include 1% to 5% lysine, 0.5% to 2% arginine malate, 0.25% to 1% arginine alpha ketoglutarate, 1% to 5% taurine, 0.1% to 1% magnesium sulfate, 1% to 5% multivitamins, 1% to 10′)/0 sucralose, 0.1% to 1% xanthan gum, 1% to 10′)/0 citric acid, and 0.1% to 1% potassium sorbate. Lysine from about 1 mg to about 5 mg, arginine malate from about 0.5 mg to about 2 mg, arginine alpha ketoglutarate from about 0.25 mg to about 1 mg, taurine from about 1 mg to about 5 mg, magnesium sulfate from about 0.1 mg to about 1 mg, multivitamins from about 1 mg to about 5 mg, sucralose from about 1 mg to about 10 mg, xanthan gum from about 0.1 mg to about 1 mg, citric acid from about 1 mg to about 10 mg, and potassium sorbate from about 0.1 mg to about 1 mg may be present per 100 ml of the drink composition.

The method of the invention for relieving fatigue and restoring energy involves treating a subject suffering from fatigue with the above drink composition. Long-lasting fatigue and/or pain may be relieved in a patient suspected of having or known to have chronic fatigue syndrome, fibromyalgia, multiple sclerosis, mononucleosis, or post-viral fatigue.

### DESCRIPTION OF SPECIFIC EMBODIMENTS

Cannabis compounds can be synthetic (chemically synthesized) or extracted from Cannabis plants such as Cannabis sativa, Cannabis indica, hemp, or hybrid strains of Cannabis sativa and Cannabis indica. A preferred source of cannabidiol (CBD) is so-called organic CBD, which is extracted from Cannabis and contains minor amounts of other cannabinoids including THC (e.g., less than 1% by dry weight).

Suitable pharmaceutically acceptable Cannabis components include Cannabis extract, which includes phytocannabinoids such as tetrahydrocannabinol “THC” (9-tetrahydrocannabinol (Δ-9 THC), 8-tetrahydrocannabinol (Δ-8 THC) and 9-THC acid), cannabidiol (CBD), other phytocannabinoids such as cannabinol (CBN), cannabichromene (CBC), cannabigerol (CBG) among others, terpenoids and flavonoids (e.g., full-spectrum CBD). Standardized Cannabis extract (SCE) consists of mostly THC, CBD and CBN. Organic CBD is extracted from organically grown Cannabis plants having lesser or trace amounts of other cannabinoids, terpenoids, and flavonoids (e.g., less than 10 ppm THC). Synthetic or pure CBD, which is free of THC and other Cannabis compounds, is a preferred Cannabis component.

THC and CBD can be extracted from a Cannabis indica dominant strain using, for example, high pressure and carbon dioxide or ethanol as a solvent in a 1500-20 L subcritical/supercritical CO₂ system made by Apeks Super Critical Systems, 14381 Blamer Rd., Johnstown, Ohio, 43031.

Another source of CBD that is essentially free of THC is the CBD mixture obtained from hemp or by extracting hemp seed oil. See Leizer et al. (The composition of hemp seed oil and its potential as an important source of nutrition. J Nutraceuticals Functional & Medical Foods 2000; 2(4):35-53). Elixinol (D&G Health LLC) is a predominantly CBD product extracted from hemp seed oil that contains trace amounts of THC.

The invention provides a method for treating fatigue, sore muscles, and pain associated therewith by administrating to a subject in need thereof a composition including: (i) an effective amount of caffeine, (ii) an effective amount of Cannabis compound such as CBD; and (iii) a third component comprising effective amounts of at least three members selected from the group consisting of lysine, arginine malate, arginine alpha ketoglutarate, taurine, niacin, magnesium sulfate, multivitamins, sucralose, vitamin C, citric acid, potassium sorbate, and xanthan gum all dissolved in reverse osmosis water, with natural flavoring.

The common mechanism underlying chronic fatigue and chronic stress is related to inflammation within the brain and peripheral nervous system. The cannabinoids found in cannabidiol (CBD) oil interact with the Endocannabinoid System by involving to the two major types of cannabinoid receptors; CB1 and CB2. These are the receptors that are found in the immune and brain cells throughout the body.

CBD oil is thought to be an energy booster. CBD oil helps in regulating normal energy levels by promoting wakefulness and ensuring that the body's cells are in good condition throughout the day (Murillo-Rodriguez et al. Curr Neuropharmacol 2014 May; 12(3):269-272).

Like CBD, another cannabinoid compound, tetrahydrocannabinol (THC), in combination with CBD has been found to be beneficial in chronic fatigue patients such as multiple sclerosis patients, but with side effects.

Another ingredient in the therapeutic drink serves as a catalyst for boosting energy and relieving stress during fatigue. These include taurine, vitamin B complex (e.g., niacin, B₆, inositol, B₁₂), arginine malate, lysine, magnesium sulfate, xanthan gum, and maltitol.

Taurine has been reported to have anti-inflammatory actions and has been suggested have health benefits (Caine & Geracioti. Taurine, energy drinks, and neuroendocrine effects. Cleveland Clin J Med 2016 December; 83(12):895-904).

Arginine malate is an essential amino acid supplement required for protein synthesis. L-arginine is found in red meat, poultry, fish, and dairy products. It can also be made in a laboratory. L-arginine used for improving athletic performance and boosting the immune system. Arginine supplements like arginine malate and arginine alpha ketoglutarate will provide similar support if used in energy drinks.

The essential amino acid, lysine is required for building body proteins. Since our bodies cannot produce/synthesize essential amino acids, including lysine. The diet is the only way to make getting the essential amino acid. Lysine supplement in energy drink will provide essential nutrient to sports persons.

Magnesium sulfate is a common mineral pharmaceutical preparation of magnesium, commonly known as Epsom salt and used both externally and internally. Magnesium is very essential mineral for the normal functioning of cells, nerves, muscles, bones, and heart.

Xanthan gum is a sugar-like compound and used as a saliva substitute to reduce dry mouth.

Maltitol is a sugar alcohol used as a sugar substitute. It has 75-90% of the sweetness of sucrose (table sugar) and nearly identical properties. It is used to replace table sugar because it has half as caloric value. Another sugar alcohol is erythritol. Aspartame, saccharine, stevia, and sucralose are artificial sweeteners.

The natural flavoring(s) may be one or more essential oils, oleoresins, essences, solvent extractives, hydrolysates, distillates, and other products of roasting, heating, or enzymatic hydrolysis, which contains the flavoring constituents derived from a spice, herb, or fruit (e.g., berries and citrus).

Most of the individuals who suffer from fatigue also have one or two other symptoms like joint and muscle pain, poor concentration, headache, and disturbed sleep. The mechanism of fatigue is often unknown.

While the cause of chronic fatigue is unknown, there are many models, ranging from psychological stress to viral infections or over exertion. It is also thought that neuroendocrine dysfunction and environmental toxins could cause complications. Many experts believe that chronic fatigue can be brought on by a combination of these factors.

The underlying symptoms of chronic fatigue involves lack of energy, lack of motivation and associated with body aches. The physiological underlying mechanism involves demand of energy molecule during fatigue. Secondly, inflammation plays role in promoting muscle soreness, body aches and feeling of tiredness (McFarlin et al. BBA Clin 2016 June; 5:72-78).

Fatigue is a physiological symptom is associated with normal physiological exertion due to sports, gym, running, hard labor among others. Chronic fatigue is often associated with disease conditions like multiple sclerosis, Alzheimer's disease, Parkinson disease, viral infections (e.g., Epstein-Bar virus, cytomegalovirus, human herpesvirus 6, enteroviruses, dengue virus, West Nile virus, and human immunodeficiency virus), and other chronic complaints. Fatigue is associated with muscle soreness, body pain, and a feeling of tiredness.

Unlike other energy drinks, the invention avoids the use of added sugars but contains caffeine, non-nutritive stimulants (e.g., guarana, ginseng, yerba mate, taurine, L-carnitine, D-glucuronolactone, and inositol) and certain vitamins and minerals (e.g., niacin, vitamin B₆ such as pyridoxine, vitamin B₁₂ such as cyanocobalamin, vitamin C).

Guarana is a plant extract and is native to South America. Guarana contains 40 mg of caffeine in 1 g of guarana. Guarana is often included as an ingredient in energy drinks for its stimulatory effect (Scholey & Haskell-Ramsay. Neurocognitive effects of guarana plant extract. Drugs Future 2008 October, 33(10):869).

High doses of caffeine can lead to anxiety, insomnia, digestive issues, high blood pressure, and fatigue, among others. High doses of THC can lead to dry mouth, high blood pressure, nausea, hallucination, dizziness, and headache, among others. High doses of CBD can result in undesirable side effects such as dry mouth, low blood pressure, light headedness, and drowsiness among others.

The combination of a low dose of caffeine and THC unexpectedly leads to enhanced mitochondrial function without side effects.

The combination of a low dose of caffeine and CBD unexpectedly leads to enhanced mitochondrial function without side effects.

The combination of a low dose of caffeine and Cannabis oil unexpectedly leads to enhanced mitochondrial function without side effects.

The combination of a low dose of caffeine and Cannabis oil unexpectedly leads to enhanced mitochondrial function without side effects.

The combination of a low dose of caffeine and THC and CBD unexpectedly leads to enhanced mitochondrial function without side effects.

The combination of a low dose of caffeine and THC unexpectedly leads to relief from fatigue, faster recovery time, faster relief from sore muscles and relief from associated pain with minimal side effects.

The combination of a low dose of caffeine and CBD unexpectedly leads to relief from fatigue, faster recovery time, faster relief from sore muscles and relief from associated pain with minimal side effects.

The combination of a low dose of caffeine with Cannabis oil unexpectedly leads to relief from fatigue, faster recovery time, faster relief from sore muscles and relief from associated pain with minimal side effects.

The combination of a low dose of caffeine with hemp oil unexpectedly leads to relief from fatigue, faster recovery time, faster relief from sore muscles and relief from associated pain with minimal side effects.

The combination of a low dose of caffeine with THC and CBD unexpectedly leads to relief from fatigue, faster recovery time, faster relief from sore muscles and relief from associated pain with minimal side effects, or any combination thereof.

The guarana plant, including extracts or powders from the seed, is a preferred source of caffeine, combined with THC, or CBD, or THC and CBD has the advantage and novelty of being very low in sugar and thereby decreasing many of the side effects associated with energy drinks that contain high amounts of sugar. Solvent extraction and/or grinding into powder may also be used with other sources of caffeine such as coffee or cocoa beans, kola nuts, and camellia leaves.

This unique combination of caffeine with CBD or THC or hemp oil, or Cannabis oil result in synergetic effect on mitochondrial function as well as on anti-inflammatory effects. Mitochondria is present in all cells including muscles as well as brain cells. A study had demonstrated that a low concentration of THC promotes enhancement of mitochondrial function in Alzheimer's cells (Cao et al. J Alzheimers Dis 2014; 42(3):973-984). However, this has not been shown for chronic or general fatigue. CBD also plays an important role in decreasing oxidative stress, enhancing mitochondrial bioenergetics and regulating glucose metabolism as well as preserving both energy and redox balance. (Sun et al. Redox Biol 2017 April; 11:577-585).

The presence of taurine and cannabinoids are anti-inflammatory in nature and relieve pain. Presence of xanthine gum relieves the dry mouth due to exercise, sports, gym or side effects of medications in older subjects.

Nanomolar concentrations of an essential amino acid such as lysine, helps with protein synthesis. This provides a critical support for muscle build up and muscle protein synthesis during muscle soreness and fatigue. Lysine further aids in collagen formation and promotes good bone health by increasing calcium absorption.

The formulation may also contain micro ingredients such as di-arginine malate and arginine α-ketoglutarate. Di-arginine malate is a precursor for nitric oxide synthesis all of which help with muscle relaxation. Arginine α-ketoglutarate is involved in the activation of nitric oxide synthase, which helps in the formation of nitric oxide. Both ingredients are essential for blood circulation, healing, and relaxes muscle fatigue. Nitric oxide also promotes muscle endurance and muscle strength.

Inclusion of low nanomolar concentrations of magnesium sulfate to the energy drink helps in the normal functioning of cells, nerve cells, and bone cells. Alternative magnesium salts that are water soluble include magnesium chloride, magnesium aspartate, magnesium glucinate, magnesium glycinate, magnesium lactate, magnesium malate, magnesium orotate, and magnesium L-threonate.

In addition, we include micromolar concentration of the sugar substitute maltitol, which replaces table sugar and has half its caloric content. The addition of nanomolar concentrations of multi-vitamins to the drink will aid in reducing body stress.

Potassium sorbate in the drink aids in preserving cannabinoids like CBD or THC as well as other ingredients and enhances their stability in the solution. Alternative stabilizers include citric acid, sodium sorbate, sodium benzoate, and potassium benzoate.

CBD, THC, hemp. and Cannabis oil are insoluble in water due to hydrophobic in nature of the compounds. We utilize nano emulsion technologies (Heilscher. Ultrasonic production of nano-size dispersions and emulsions. ENS 2005 December, 138-143) to dissolve these compounds. This technology involves Nano emulsification of mixing hydrophobic compounds with water. Several other known techniques are also available to dissolve CBD, THC, or hemp and Cannabis oil in water.

A similar methodology is used with other caffeine rich substances like coffee, cocoa, kola nut, or tea that may be substituted for guarana.

Another method for drinking a beverage (e.g., energy drink) is administering to a subject in need thereof the beverage comprising (i) an effective amount of caffeine and (ii) an effective amount of a Cannabis compound such as CBD, THC, CBN, CBC, CBG, or a combination of two or more such compounds (e.g., CBD and/or THC). They are formulated as an aqueous solution together with optional, generally recognized as safe (GRAS) ingredients, such as those described above, preferably as a sugar-free beverage. The beverage is then packaged as an individual serving of from about 200 ml to about 400 ml (e.g., 250 ml) in an aluminum can or plastic bottle.

Yet another method for drinking a beverage (e.g., energy drink) is administering to a subject in need thereof the beverage comprising (i) an effective amount of caffeine and (ii) an effective amount of hemp oil, Cannabis oil, full-spectrum CBD oil, broad-spectrum CBD oil, or CBD isolate. They are formulated as an aqueous solution together with optional GRAS ingredients, such as those described above, preferably as a sugar-free beverage. The beverage is packaged as an individual serving of from about 200 ml to about 400 ml (e.g., 250 ml) in an aluminum can or plastic bottle.

For example, GRAS ingredients may include arginine supplements (e.g., arginine malate and arginine alpha ketoglutarate), taurine, lysine, vitamins (e.g., niacin and vitamin C), magnesium sulfate, xanthan gum, stabilizers (e.g., citric acid and potassium sorbate), and natural flavoring. Instead of sucrose or glucose, sugar substitutes (e.g., sucralose and maltitol) are added in sugar-free beverages.

All publications cited above are incorporated by reference at the place where cited and for particular disclosures that are relevant to their descriptions of specific embodiments at those places. The following examples are merely illustrative.

### Example 1

A combination of 25% Guarana extract and 0.25% THC is combined in 100 ml reverse osmosis water along with range percentages of 1% to 5% taurine, 1% to 5% lysine, 0.5% to 2% arginine malate, 0.25% to 1% arginine α-ketoglutarate, 1% to 5% vitamin B complex, and 1% to 5% niacin then administrated to a female adult age 27 who has fatigue from mountain climbing. Prior to administration of the formulation, the individual scored an 8 on the Visual Analogue Scale to Evaluate Fatigue Severity (VAS-F). The scale is from 1 to 10 where 10 represents extremely fatigued. After the administration of 100 ml of the formulation, the individual scores a 4 on the VAS-F scale, after about 30 minutes.

### Example 2

The formulation containing a combination of 25% Guarana extract and 5% CBD is combined in 100 ml reverse osmosis water along with micromolar concentrations of 1% to 5% taurine, 1% to 5% lysine, 0.5% to 2% arginine malate, 0.25% to 1% arginine α-ketoglutarate, 1% to 5% vitamin B complex, and 1% to 5% niacin then administrated to a patient suffering from Parkinson's disease.

The individual is a male adult age 73 who suffers from fatigue. Prior to administration of the formulation, the individual scored a 9 on the Visual Analogue Scale to Evaluate Fatigue Severity (VAS-F). The scale is from 1 to 10 where 10 represents extremely fatigued. After the administration of 100 ml of the formulation, the individual scored a 5 on the VAS-F scale after about 30 minutes.

While this invention may be described in terms of preferred chemical components, amounts or concentrations, ranges or ratios of quantities, functional activities of each components or individual constituents thereof, steps and their order for methods of treatment or manufacturing processes, reaction parameters or conditions, tests and assays, performance protocols, designs, materials and structures of devices, it is understood that the invention is capable of further modifications, uses and/or adaptations following in general the principle taught herein, and including such departures from the present disclosure as those that come within the known or customary practice in the art to which the invention pertains, and as may be applied to the central features hereinbefore set forth, which fall within the scope of the invention and of the limits of the claims appended hereto or presented later. Therefore, the legal protection of the invention is not limited to any of the specific embodiments described herein unless their certain features are recited in those claims.

1. A therapeutic drink composition which is dissolved in reverse osmosis water with natural flavoring and comprises: (i) a first component selected from the group of cannabidiol (CBD), tetrahydrocannabinol (THC), hemp oil, Cannabis oil, and CBD together with THC; (ii) caffeine; and (iii) a third component comprising at least three members selected from the group consisting of lysine, arginine malate, arginine a ketoglutarate, taurine, niacin, magnesium sulfate, multivitamins, vitamin C, sucralose, citric acid, potassium sorbate, and xantham gum.

2. The composition of claim 1 wherein the caffeine is from a guarana extract.

3. (canceled)

4. The composition of claim 1 wherein the CBD is in the range 0.5% to 10%.

5. The composition of claim 1 wherein the THC is in the range 0.0025% to 2%.

6. The composition of claim 1 wherein the caffeine is in the range 0.01% to 0.8%.

7. The composition of claim 1 wherein the lysine is in the range 1% to 5%.

8. The composition of claim 1 wherein the arginine malate is in the range 0.5% to 2%.

9. The composition of claim 1 wherein the arginine a ketoglutarate is in the range 0.25% to 1%.

10. The composition of claim 1 wherein the taurine is in the range 1% to 5%.

11. The composition of claim 1 wherein the magnesium sulfate is in the range 0.1% to 1%.

12. The composition of claim 1 wherein the multivitamins are in the range 1% to 5%.

13. The composition of claim 1 wherein the citric acid is in the range 1% to 10%.

14. The composition of claim 1 wherein the sucralose is in the range 1% to 10%.

15. The composition of claim 1 wherein the potassium sorbate is in the range 0.1% to 1%.

16. The composition of claim 1 wherein the xanthan gum is in the range 0.1% to 1%.

17. A method for relieving fatigue, restoring energy, rejuvenating sore muscles, relieving pain, or any combination thereof which comprises administering to a subject in need thereof an effective amount of the composition of claim 1.

18. (canceled)

19. A method for relieving fatigue and restoring energy which comprises administering the composition of claim 1 to a subject suffering from fatigue, sore muscles, or pain.
